(19) United States
(12) Patent Application Publication
(10) Pub. No.: US 2007/0093499 A1
(43) Pub. Date: Apr. 26, 2007

(54) Use of dasatinib for the treatment of bone metastasis

(75) Inventors: Francis Y. Lee, Yardley, PA (US); Feng Roger Luo, Plainsboro, NJ (US); Jean H. M. Feyen, New Hope, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/583,564

(22) Filed: Oct. 19, 2006

Related U.S. Application Data
(60) Provisional application No. 60/728,731, filed on Oct. 20, 2005.

(51) Int. Cl. A61K 31/506

(52) U.S. Cl. 514/252.19

(57) ABSTRACT

The present disclosure is directed to a method of treating bone metastasis, hypercalcemia, and/or bone resorption comprising administering to a patient in need thereof a therapeutically effective amount of a compound of formula (I) 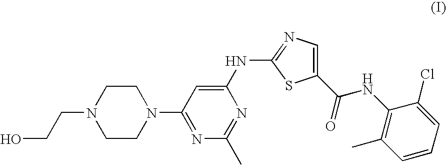 or pharmaceutically acceptable salt, hydrate or solvate thereof.

### RELATED APPLICATION

This application claims priority benefit under Title 35 §119(e) of U.S. provisional Application No. 60/728,731, filed Oct. 20, 2005, the contents of which are herein incorporated by reference.

The invention relates to the use of a protein tyrosine kinase inhibitor in the treatment of bone metastasis.

Bone metastases are a frequent complication of malignancy, occurring in the majority of patients with advanced breast and prostate cancer and multiple myeloma, as well as between 15-30% of patients with cancers of the lung, colon, stomach, bladder, uterus, thyroid and kidney (Coleman, R. E. and R. D. Rubens (1987). “The clinical course of bone metastases from breast cancer.” Br J Cancer 55(1): 61-6.). It is estimated that each year in the United States 350,000 people die with bone metastases (Mundy, G. R. (2002). “Metastasis to bone: causes, consequences and therapeutic opportunities.” Nat Rev Cancer 2(8): 584-93.). Bone metastases have been characterized as osteolytic and osteoblastic, both of which frequently cause intractable bone pain, pathological fractures, life-threatening hypercalcemia and various nerve compression syndromes (Roodman, G. D. (2004). “Mechanisms of bone metastasis.” N Engl J Med 350(16): 1655-64.). Currently, there are few effective therapies for either preventing or inhibiting the development of metastases to bone.

The osteolytic bone resorption is mediated by activated osteoclasts (Kodama, H., A. Yamasaki, et al. (1991). “Congenital osteoclast deficiency in osteopetrotic (op/op) mice is cured by injections of macrophage colony-stimulating factor.” J Exp Med 173(1): 269-72; Clohisy, D. R., S. L. Perkins, et al. (2000). “Review of cellular mechanisms of tumor osteolysis.” Clin Orthop Relat Res (373): 104-14). The proliferation and stimulation of osteoclasts is strongly associated with production of osteoclastogenic factors such as macrophage-colony stimulating factor (M-CSF) by tumor cells (Clohisy, Perkins et al. 2000). Binding of M-CSF to its receptor CSF-1R (c-fins gene product) on monocytic progenitors stimulates formation and osteolytic function of osteoclasts (Kodama, Yamasaki et al. 1991; Insogna, K. L., M. Sahni, et al. (1997). “Colony-stimulating factor-I induces cytoskeletal reorganization and c-src-dependent tyrosine phosphorylation of selected cellular proteins in rodent osteoclasts.” J Clin Invest 100(10): 2476-85; Feng, X., S. Takeshita, et al. (2002). “Tyrosines 559 and 807 in the cytoplasmic tail of the macrophage colony-stimulating factor receptor play distinct roles in osteoclast differentiation and function.” Endocrinology 143(12): 4868-74). In addition to a critical role in osteoclast development, CSF-1R also contributes to tumorigenesis. Transgenic mice susceptible to mammary cancer development crossed with osteopetrotic mice lacking M-CSF and osteoclast production have decreased development of late stage invasive carcinoma and pulmonary metastasis (Kodama, Yamasaki et al. 1991; Insogna, Sahni et al. 1997; Feng, Takeshita et al. 2002). CSF-1R expression has been detected in human cancer cells including breast (Kacinski, Scata et al. 1991; Kacinski 1995) and has been correlated with poor prognosis as well as invasive potential (Tang, R., F. Beuvon, et al. (1992). “M-CSF (monocyte colony stimulating factor) and M-CSF receptor expression by breast tumour cells: M-CSF mediated recruitment of tumour infiltrating monocytes?” J Cell Biochem 50(4): 350-6; Maher, M. G., E. Sapi, et al. (1998). “Prognostic significance of colony-stimulating factor receptor expression in ipsilateral breast cancer recurrence.” Clin Cancer Res 4(8): 1851-6). Taking all experimental evidence into account, a therapeutic agent targeting CSF-1R and osteoclast activity could offer clinical benefits to breast cancer patients with metastatic disease. A proof of principal study with a CSF-1R inhibitor Su-11248 has demonstrated the inhibitory potency on the formation and function of osteoclasts in cell culture as well as on bone resorption in vivo (Murray, L. J., T. J. Abrams, et al. (2003). “SU11248 inhibits tumor growth and CSF-1R-dependent osteolysis in an experimental breast cancer bone metastasis model.” Clin Exp Metastasis 20(8): 757-66).

Strong evidence exists linking SRC kinase activation with tumor progression and metastasis. Tumor cells with heightened metastatic potential have been shown to have activated SRC kinase (Mao, W., R. Irby, et al. (1997). “Activation of c-Src by receptor tyrosine kinases in human colon cancer cells with high metastatic potential.” Oncogene 15(25): 3083-90). In epithelial tumors, SRC promotes the metastatic phenotype by disrupting and/or weakening the normally strong cell-cell adhesions, increasing the cell-matrix interaction, and enhancing cell migration (Avizienyte, E., A. W. Wyke, et al. (2002). “Src-induced de-regulation of E-cadherin in colon cancer cells requires integrin signaling.” Nat Cell Biol 4(8): 632-8; Frame, M. C., V. J. Fincham, et al. (2002). “v-Src's hold over actin and cell adhesions.” Nat Rev Mol Cell Biol 3(4): 233-45). In addition to the role in tumor growth and metastasis, several recent studies also linked SRC kinase with osteoclast formation, function, and osteolytic bone resorption (Tanaka, S., M. Amling, et al. (1996). “c-Cb1 is downstream of c-Src in a signaling pathway necessary for bone resorption.” Nature 383(6600): 528-31). It is of great interest and importance that the activation of SRC kinase has found to be mediated through the CSF-1R signaling pathway upon stimulation by CSF-1 in osteoclasts (Insogna, K., S. Tanaka, et al. (1997). “Role of c-Src in cellular events associated with colony-stimulating factor-1-induced spreading in osteoclasts.” Mol Reprod Dev 46(1): 104-8). A selective SRC kinase inhibitor, CGP77675, has demonstrated the inhibitory potency on parathyroid hormone (PTH)-induced bone resorption in fetal rat long bone culture as well as in ovariectomized rats (Missbach, M., M. Jeschke, et al. (1999). “A novel inhibitor of the tyrosine kinase Src suppresses phosphorylation of its major cellular substrates and reduces bone resorption in vitro and in rodent models in vivo.” Bone 24(5): 437-4).

The compound of formula (I) N-(2-chloro-6-methylphenyl)-2-((6-(4-(2-hydroxyethyl)-1-piperazinyl)-2-methyl-4-pyrimidinyl)amino)-1,3-thiazole-5-carboxamide, (also known as BMS-354825 and dasatinib) is a protein tyrosine kinase inhibitor and is a Src Kinase inhibitor and is useful in the treatment of oncological and immunologic diseases. The compound of formula (I) is also known as dasatinib and BMS-354825. The compound of formula (I) is also an inhibitor of BCR/ABL, and/or ABL inhibitor. Compounds which inhibit Src and/or BCR/ABL are useful in the treatment of cancers such as CML and ALL.

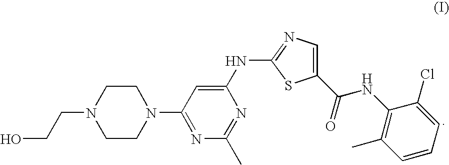

The compound of formula (I) and its preparation have been previously described in U.S. Pat. No. 6,596,746, issued Jul. 22, 2003. The compound is ideally a crystalline monohydrate form such as described in U.S. patent application Ser. No. 11/051,208, filed Feb. 4, 2005, which is now U.S. Publication No. US20050215795A1, published Sep. 29, 2005 and U.S. patent application Ser. No. 11/192,867, filed Jul. 29, 2005, which is now U.S. Publication No. US20060004067A1, published Jan. 5, 2006, which are hereby incorporated by reference. Alternatively, the compound of formula (I) may exist in other crystalline forms, either as a neat compound or as a solvate as described in the applications described above. A pharmaceutical composition of the compound is described in U.S. Patent Application Ser. No. 60/678,030, filed May 5, 2005, which is now U.S. patent application Ser. No. 11/418,338, filed May 4, 2006, which are hereby incorporated by reference.

### BRIEF DESCRIPTION OF THE DRAWINGS

FIG. 1 shows the inhibitory effects of BMS-354825 at various concentrations on the formation of TRAP positive osteoclasts in mouse bone marrow cell culture in the presence of CSF and RANK ligand.

FIG. 2 shows quantitation of the inhibition of osteoclast formation (A) and inhibitory potency of BMS-354825 (B) in mouse bone marrow cell culture. TRAP Positive TRAP staining cells with multiple nuclei (≧3) were counted as osteoclasts.

FIG. 3 shows the inhibition of serum levels of calcium in TPTX rats administered BMS-354825 orally.

FIG. 4 shows a comparison of inhibition of serum levels of calcium in TPTX rats between BMS-354825 and zometa.

FIG. 5 shows pharmacokinetics of BMS-354825 on day 1 in TPTX following a multiple treatment schedule QD×5 orally. Each point represents the mean (±SD) for at least three observations.

FIG. 6 shows the correlation of pharmacokinetics and pharmacodynamics of BMS-354825 in TPTX rats. Each point represents the mean (±SD) for at least three observations.

FIG. 7 shows the ability of BMS-354825 to inhibit the release of radiolabeled calcium (45Ca) from bone in vitro.

FIG. 8 shows the ability of BMS-354825 to inhibit normalization of serum calcium after infusion of PTH in thyro-parathyroidectomized rats.

### SUMMARY OF THE INVENTION

It has been found that the compound of formula (I) is a dual inhibitor of Src and CSF-1R kinases. The compound of formula I is therefore useful in the treatment of tumor bone metastasis as well as related bone resorption and hypercalcemia.

In one embodiment, there is disclosed a method of treating bone metastasis which comprises administering to a mammalian specie in need thereof a therapeutically effective amount of the compound of formula (I), pharmaceutically acceptable salt, hydrate or solvate thereof.

In another embodiment, there is disclosed a method of inhibiting hypercalcemia which comprises administering to a mammalian specie in need thereof a therapeutically effective amount of the compound of formula (I), pharmaceutically acceptable salt, hydrate or solvate thereof.

In another embodiment, there is disclosed a method of inhibiting bone resorption which comprises administering to a mammalian specie in need thereof a therapeutically effective amount of the compound of formula (I), pharmaceutically acceptable salt, hydrate or solvate thereof.

In another embodiment, there is disclosed a method of treating hypercalcemia (both cancer related and non-related) and bone metastasis, which comprises administering to a mammalian specie in need thereof a therapeutically effective amount of the compound of formula (D, pharmaceutically acceptable salt, hydrate or solvate thereof.

In another embodiment, there is disclosed a pharmaceutical composition for the treatment of bone metastasis, hypercalcemia and/or bone resorption which comprises the compound of Formula I, and a pharmaceutically acceptable carrier.

In another embodiment, there is provided a use of the compound of formula (I) in the therapy.

In another embodiment, there is provided a use of the compound of formula (I) in the preparation of a medicament for the treatment of bone metastasis, hypercalcemia and/or bone resorption.

In another embodiment, there is provided a use of the compound of formula (I) in the preparation of a medicament for the treatment of bone metastasis, hypercalcemia and/or bone resorption for cancer patients.

In another embodiment, there is provided a use of the compound of formula (I) in the preparation of a medicament for the treatment of bone metastasis, hypercalcemia and/or bone resorption for breast cancer patients.

The invention may be embodied in other specific forms without departing from the spirit or essential attributes thereof. This invention also encompasses all combinations of alternative aspects and embodiments of the invention noted herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

### DETAILED DESCRIPTION OF THE INVENTION

### DEFINITIONS

As used herein, the term “breast cancer patient” is a patient having breast cancer and being treated by the disease.

As used herein, the term “Bone metastasis”, includes, but is not limited to, the spread of a cancer to a new part of the body is called metastasis. Bone is one of the most common site of metastatic spread. Many people with cancer (except for those with non-melanoma skin cancer) develop bone metastasis at some point in the course of their disease. Breast, prostate, lung, kidney and thyroid cancers and some blood cancers (e.g. multiple myeloma) are most likely to spread to bones.

As used herein, the term “Hypercalcemia”, includes, but is not limited to, a disorder in which the level of calcium in the blood is too high. Hypercalcemia is the most common life-threatening disorder associated with cancer.

As used herein, the term “Bone resorption” includes, but is not limited to, the process of bone breakdown and the release of bone minerals (calcium, magnesium, phosphate and by-products of collagens) from bone fluid to the blood.

As used herein, the term “Osteoclasts”, includes, but is not limited to, the following description. Bone resorption is the unique function of the osteoclasts, which are specialized, macrophage polykaryon (multi-nucleated cells) that contain numerous mitochondria and lysosomes. The osteoclast possesses a specialized cytoskeleton that upon SRC kinase signaling permits it to establish an isolated microenvironment between itself and bone, wherein matrix degradation occurs by a process involving proton transport.

Osteoclastogenesis—The formation of osteoclasts. Osteoclastogenesis is principally regulated by macrophage colony-stimulating factor (CSF-1), RANK ligand, and osteoprotegerin.

### Methods

Unless otherwise specified, all chemicals and reagents were obtained from Sigma (St. Louis, Mo.). Sterile buffers and solutions were obtained from Invitrogen (Carlsbad, Calif.). Sterile tissue culture ware was obtained from Fisher Scientific Co. (Hanover Park, Ill.). DMEM (cat #11995-040) and Fetal Bovine Serum (FBS) (cat #16140-071) were obtained from Invitrogen (Carlsbad, Calif.). mCSF-1 (cat #416-ML) and receptor activator for nuclear kappaB (RANK) ligand (cat#462-TR) cytokines were purchased from R&D Systems, Inc (Minneapolis, Minn.). Acid Phosphatase, Leukocyte staining kit (cat #387-A) was purchased from Sigma (St. Louis, Mo.) Parathyroid Hormone human 1-34 (PTH) (cat #P3796) and thyrocalcitonin (cat #T3660) were purchased from Sigma (St. Louis, Mo.). Zometa (NDC#0078-0387-25) was purchased from Novartis Pharmaceuticals (East Hanover, N.J.). Alzet®mini pumps (cat #1007D) were purchased from Durect Corporation (Cupertino, Calif.). Isoflurane (NDC#10019-773-60) was purchased from Baxter Pharmaceuticals (Deerfield, Ill.).

Female CDF-1 mice, 5-6 weeks of age, were obtained from Harlan Sprague-Dawley Co (Indianapolis, Ind.), and maintained in an ammonia-free environment in a defined and pathogen-free colony. Animals were quarantined for approximately 3 weeks prior to their use for tumor propagation and drug efficacy testing. Male Sprague-Dawley rats (175-200 gram body weight) were received thyroparathyroidectomized (TPTX) from Taconic (Germantown, N.Y.) and were delivered to the animal facility two days post surgery. TPTX Rats were quarantined for approximately 13 days prior to their use. Animals were provided with food and water ad libitum. All studies were performed in accordance with Bristol-Myers Squibb (BMS) and the American Association for Accreditation of Laboratory Animal Care (AAALAC).

For oral (PO) administration, BMS-354825 was dissolved in 80 mM citrate buffer. The volume of administration for BMS-354825 was 0.01 ml/gm for mice and 0.005 ml/gm for rats. For subcutaneous (SC) administration, Zometa was diluted in citrate buffer and administered at 0.005 ml/gm for rats. Thyrocalcitonin was diluted in 5% Dextrose and administered at 1 ug/rat.

Bone marrow was harvested by flushing the femur and tibia bones of CDF-1 mice with PBS followed by washing twice with PBS and resuspending in DMEM with 10% FBS (1-2×10⁶ cells/m, 10 ml). Cells were then seeded in 24-well plates (2×10⁶ cells/well/ml) and were cultured for 6-9 days in DMEM media supplemented with the cytokines: 10 ng/ml recombinant mouse M-CSF-1 and 100 ng/ml recombinant mouse RANK ligand to induce osteoclast development. Culture medium was replaced every two days till day 5 and there was no medium replacing between day 6 and day 9. BMS-354825 was typically dosed on day 5. On days 5, 7, and 9, cell viability and number of TRAP positive cells were determined using the Acid Phosphatase Leukocyte staining kit. The bone marrow cells were washed with PBS and stained according to the protocol recommended by the vendor. TRAP positive cells and osteoclasts were counted using an inverted microscope.

Acute Bone Resorption Inhibition in Rats in vivo

Male Sprague-Dawley TPTX rats with basal serum calcium levels around 7-8 mg/dl were randomly assigned to the experimental groups. Alzet® mini-pumps containing a resorption stimulus (PTH: infusion rate of 0.3 μg/hr) were implanted subcutaneously. In brief, rats were anesthetized with isoflurane (5% of oxygen), the skin of the flank area was disinfected with a topical disinfectant. Following a small incision, a subcutaneous pouch was made with a sterile pair of blunt scissors, a mini-pump was inserted and the incision was closed with a wound clip. Upon drug administration, 500 μl of blood was sampled at the indicated time points by retro-orbital bleeding, the serum was collected and frozen at −80° C. Levels of serum calcium and drug were analyzed accordingly.

Pharmacokinetic Analysis

The serum levels of drug were analyzed by high performance liquid chromatography/mass spectrometry (HPLC/MS). In brief, serum samples (50 μl) were de-proteinized with three volumes of acetonitrile containing 5 μg/ml of BMS-357990, wherein BMS-357990 is the compound of formula (II) below, as an internal standard (IS). After centrifugation to remove precipitated proteins, 5 μl of the supernatant was analyzed by HPLC/MS. The HPLC column was a Phenomenex Prodigy C18-ODS3 column (2 mm×50 mm, 3 μM particles) maintained at 60° C. with a flow rate of 0.5 ml/min. The mobile phase consisted of 5 mM ammonium formate pH 3.75 (A) and acetonitrile (B). The initial mobile phase composition was 87.5% of A/12.5% of B. After sample injection, the mobile phase was changed to 37.5% of A/62.5% of B over 2 minutes, and was held at that composition for an additional 1.5 minutes. The HPLC was interfaced to a Finnigan LCQ Advantage ion-trap mass spectrometer operated in the positive ion electrospray and full MS/MS mode. For BMS-354825, fragmentation of m/z 488 yielded a daughter ion for quantitation at m/z 401. For the internal standard, m/z 444 was fragmented to yield a daughter ion at m/z 303. The retention times for BMS-354825 and the IS were 3.10 and 2.75 min, respectively. The standard curve ranged from 0.004 μM to 16 μM and was fitted with a quadratic regression weighted by reciprocal concentration (1/×). The limit of quantitation (LOQ) for the purposes of this assay was 0.004 μM. Quality control (QC) samples at two levels in the range of the standard curve were used to accept individual analytical sets.

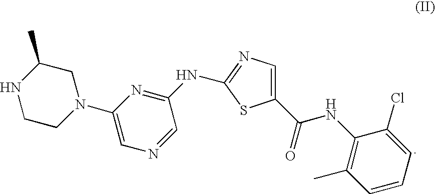

PK data analysis was performed by noncompartmental method using Kinetica (v4.0.2, InnaPhase Corporation, Philadelphia, Pa.). The maximum plasma concentration (Cₘₐₓ) and the time reaching Cₘₐₓ (Tₘₐₓ) were determined by visually inspecting the profiles of plasma level of drug vs. time. The half life of plasma drug elimination (t_{1/2}) was the ratio of 0.693 to the slope obtained by log-linear regression of the terminal phase of the drug plasma profile. The area under the plasma drug concentration curve (AUC) was estimated by the trapezoidal rule.

Determination of Serum Levels of Calcium

The serum levels of calcium of TPTX rats were quantitated by a photometric assay with the Roche Hitachi 917 (H9 17) automated chemistry analyzer (Indianapolis, Ind.). In this method, calcium reacts with o-cresolphthalein complexone in the presence of 8-hydroxyquinoline to form a purple chromophore. The color intensity of the purple complex is directly proportional to the calcium concentration and is measured photometrically on the analyzer. This method is capable of quantifying 0.2-18.2 mg/dL of serum calcium. For samples >18.2 mg/dL the samples must be diluted in saline and the value multiplied by the appropriate factor.

BMS-354825 is a potent and selective inhibitor of SRC kinase with a biochemical IC₅₀ of 0.8 nM. It also has been found to strongly inhibit CSF-1R (89% of control at 10 nM). Therefore, BMS-354825, targeting both SRC and CSF-1R kinases, is an novel method for the treatment of tumor bone metastasis as well as related bone resorption and hypercalcemia.

Osteoclast Development in Bone Marrow Cell Culture

It has been demonstrated that addition of M-CSF to murine bone marrow progenitors stimulates proliferation and initiates differentiation toward the myeloid pathway. Differentiation to osteoclasts then occurs in the presence of osteoclast differentiation factor or RANK ligand (Buckley and Fraser Buckley, K. A. and W. D. Fraser (2002). “Receptor activator for nuclear factor kappaB ligand and osteoprotegerin: regulators of bone physiology and immune responses/potential therapeutic agents and biochemical markers.” Ann Clin Biochem 39(Pt 6): 551-6.). To establish the cell culture model of osteoclasts, bone marrow cells were freshly harvested from the tibia and femur bones of mice and were seeded in 24-wells plates containing DMEM medium supplemented with 20 ng/ml M-CSF and 100 ng/ml RANK ligand, which are the conditions commonly used in murine bone marrow culturing conditions (Murray, L. J., T. J. Abrams, et al. (2003). “SU11248 inhibits tumor growth and CSF-1R-dependent osteolysis in an experimental breast cancer bone metastasis model.” Clin Exp Metastasis 20(8): 757-66.). The formation of osteoclasts was monitored by TRAP staining for 9 days. No significant TRAP positive staining cells were observed in the early phase of osteoclast development, i.e. between days 1 and 3. Starting from day 5, TRAP positive staining cells were observed, and the multi-nucleated TRAP positive staining osteoclasts (with ≧3 nuclei) were clearly detected on day 7 and became most abundant by day 9. No multi-nucleated TRAP positive staining osteoclasts were observed in control cells being cultured in the absence of CSF and RANK ligand.

Inhibition of Osteoclast Formation by BMS-354825 in Bone Marrow Cell Culture

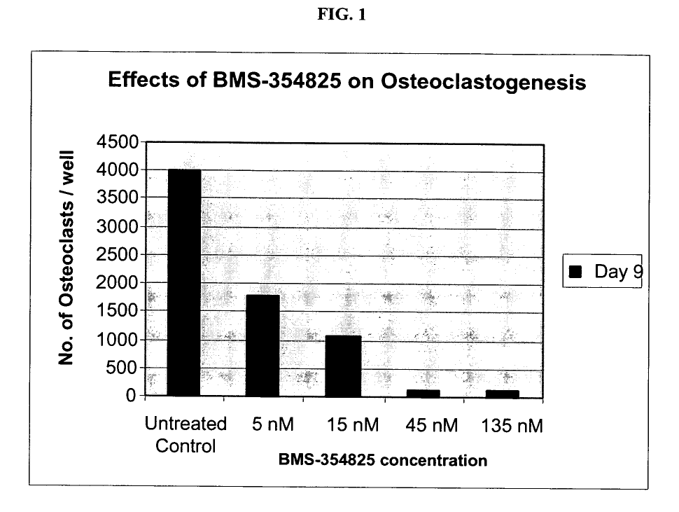

To explore the inhibition of osteoclast development, various concentrations of BMS-354825 was dosed on day 5 post cell seeding, bone marrow cells were cultured up to 9 days. BMS-354825 significantly reduced the number of the TRAP positive staining osteoclasts. It appeared that BMS-354825 was able to potently inhibit the development of osteoclasts over the entire culture period. (FIG. 1)

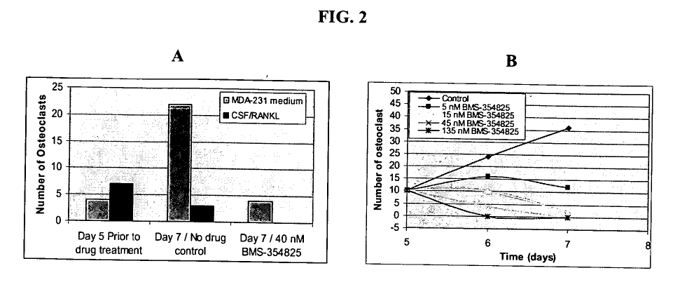

The inhibition of osteoclast development by BMS-354825 was quantitated by counting the number of the TRAP positive staining osteoclasts with multiple nuclei (≧3). It was observed that, in the absence of BMS-354825, the number of osteoclasts in MDA-MB-231 condition media was greater than that in DMEM medium supplemented with CSF/RANK ligand, indicating a greater stimulation of osteoclast development by condition medium (FIG. 2A). It is speculated that condition medium could contain more kinds and/or higher levels of cytokines/growth factors secreted by tumor cells, which in turn result in a stronger stimulatory effect on osteoclast development. When dosed with BMS-354825, the number of osteoclasts was significantly reduced for both culturing conditions (FIG. 2A).

The inhibitory effect of BMS-354825 on osteoclast development was further titrated in bone marrow cells cultured in DMEM media supplemented with M-CSF/RNAK ligand. BMS-354825 was dosed on day 5 post seeding, the formation of osteoclasts was inhibited dose dependently (FIG. 2B). The value of IC₅₀, the drug concentration required to inhibit 50% of osteoclast formation, was estimated to be 4.4 nM. This value was comparable to the inhibitory potency of bone resorption by BMS-354825 (IC₅₀ of 2 nM). It suggests that BMS-354825 is able to inhibit bone resorption likely through the inhibition of osteoclast development and function. Overall, the current data demonstrated that BMS-354825 was able to inhibit the development of osteoclasts in mouse bone marrow cell culture in vitro, which could consequently inhibit bone resorption mediated through the osteoclastogenesis pathway. We are currently exploring the mechanistic link between the inhibition of osteoclast development and the potential target inhibition by BMS-354825, i.e. SRC and/or CSF-1R kinase in osteoclasts.

Inhibition of Hypercalcemia by BMS-354825 in TPTX Rats

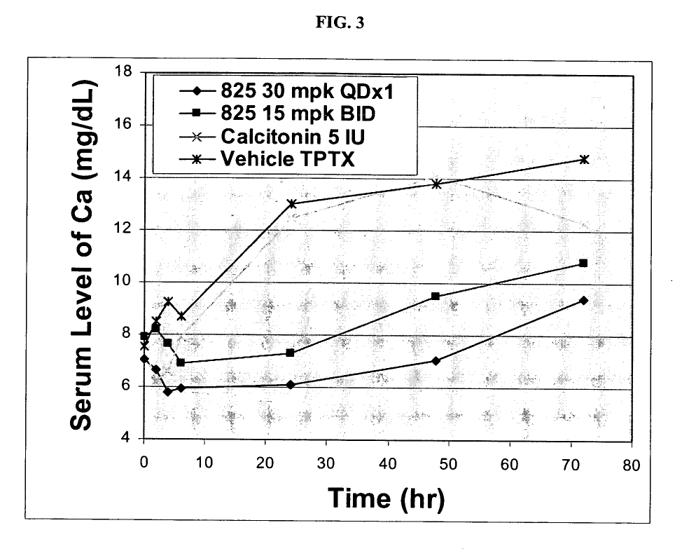

Malignancy related hypercalcemia has been strongly linked with bone resorption mediated by stimulation of native osteoclast formation and activity. BMS-354825 was therefore further tested in thyroid parathyroidectomized (TPTX) rats, a commonly used animal model to evaluate bone resorption (Fisher, J. E., M. P. Caulfield, et al. (1993). “Inhibition of osteoclastic bone resorption in vivo by echistatin, an “arginyl-glycyl-aspartyl” (RGD)-containing protein.” Endocrinology 132(3): 1411-3. Engleman, V. W., G. A. Nickols, et al. (1997). “A peptidomimetic antagonist of the alpha(v)beta3 integrin inhibits bone resorption in vitro and prevents osteoporosis in vivo.” J Clin Invest 99(9): 2284-92. Lark, M. W., G. B. Stroup, et al. (1999). “Design and characterization of orally active Arg-Gly-Asp peptidomimetic vitronectin receptor antagonist SB 265123 for prevention of bone loss in osteoporosis.” J Pharmacol Exp Ther 291(2): 612-7.). In this model, the thyroid and parathyroid are surgically removed to induce hypocalcemia, then subsequent infusion with PTH stimulates the osteoclast-mediated calcemic response. In the present study, TPTX rats with basal calcium levels from 7-8 mg/dL, were implanted subcutaneously with osmotic Alzet pumps containing PTH. Upon initiation of PTH infusion to induce hypercalcemia, TPTX rats were administered BMS-354825 at 30, 15, and 5 mpk po (QD×1). A dose dependent inhibition of serum calcium levels was observed (data not shown). Calcitonin, a drug currently being used to manage hypercalcemia clinically, was used as a reference compound at 5 IU/dose sc, and showed an inhibition of serum calcium levels, see discussion below. A study demonstrated that BMS-354825 administered orally rapidly inhibited the serum levels of calcium at 30 mpk (QD×1) as well as at 15 mpk (2QD×1), while 30 mg/kg at QD appeared more effective to inhibit serum calcium levels than 15 mg/kg at 2QD. Nonetheless, the inhibition appeared to recover with time, but the serum calcium levels of TPTX rats treated with BMS-354825 were still significantly below those of the control rats on day 3 post drug administration (FIG. 3). In the same experiment, calcitonin at 5 IU/dose sc also provided a rapid inhibition of serum calcium levels, but the inhibition was reversed rapidly. There was essentially no significant difference in serum calcium levels between the calcitonin treated and the control rats on day 3 (FIG. 3). This experiment demonstrated that BMS-354825 was able to inhibit the induction of hypercalcemia by PTH more effectively than calcitonin.

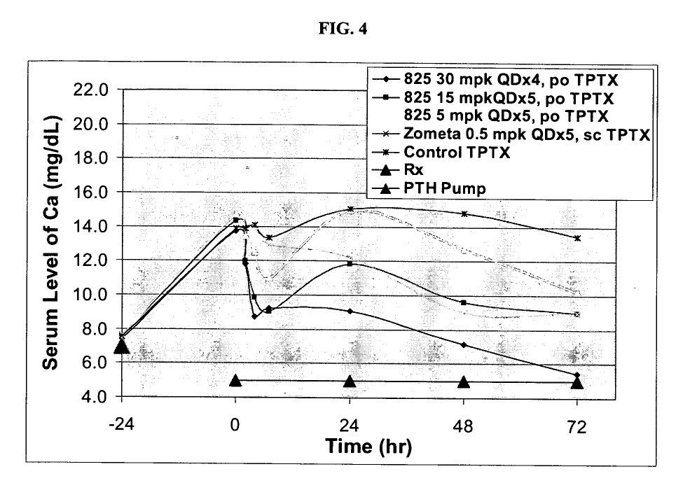

With a multiple treatment schedule, BMS-354825 was compared side by side with Zometa®, a new generation of bisphosphonates, which has demonstrated clinical benefits in breast cancer patients with bone metastases (Rosen, L. S., D. Gordon, et al. (2003). “Zoledronic acid versus placebo in the treatment of skeletal metastases in patients with lung cancer and other solid tumors: a phase III, double-blind, randomized trial—the Zoledronic Acid Lung Cancer and Other Solid Tumors Study Group.” J Clin Oncol 21(16): 3150-7). In this experiment, TPTX rats were infused with PTH for 24 hr prior to drug treatment and hypercalcemia was shown to be induced, based on the measurement of serum levels of calcium ranged from 14-15 mg/dL (FIG. 4). BMS-354825 was then administered at dose levels of 30, 15, and 5 mg/kg/dose po (QD×5) while Zometa® was administered at 0.4 mg/kg/dose sc (QD×5). Once again, BMS-354825 demonstrated a rapid inhibition of serum calcium levels within 3 hr. In contrast, zometa did not produce any appreciable inhibition until 24 hr post drug administration. The degree of inhibition of serum calcium levels by BMS-354825 at 15 mg/kg/dose was comparable to that by zometa between 24 hr and 72 hr (the end of study) post drug administration, while BMS-354825 at 30 mg/kg/dose showed more robust inhibition through the entire period of experiment. By 72 hr, the inhibition of serum calcium levels by zometa appeared similar to that by BMS-354825 at 5 mg/kg/dose which was the dose level appearing less effective with initial dosing. Due to the fact that osmotic pump works less efficiently after day 4, we intended not to include the data of calcium levels thereafter. Overall, this study demonstrated that BMS-354825 was able to inhibit the progression of hypercalcemia induced by PTH, dose-dependently. At the current once daily treatment regimen, BMS-354825 at 30 mg/kg/dose po appeared more effective than zometa at 0.4 mg/kg/dose sc, while at 15 and 5 mg/kg/dose, BMS-354825 produced comparable inhibitory effect compared to zometa at 0.4 mg/kg/dose with a multiple treatment schedule.

Pharmacokinetics of BMS-354825 in TPTX Rats Administered Orally

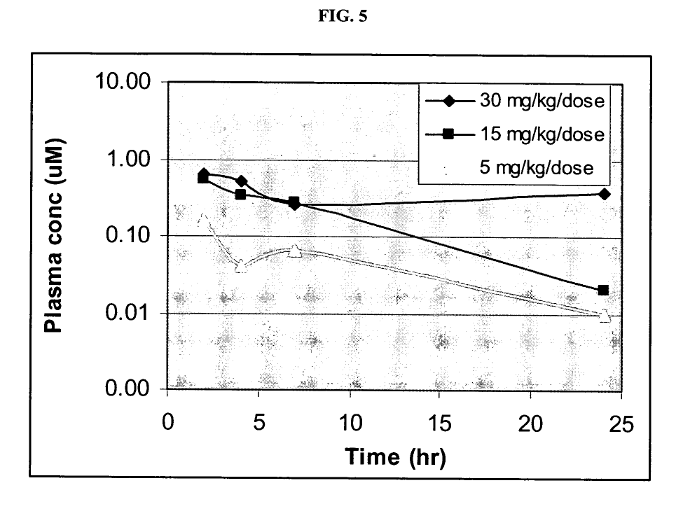

To fully characterize the relationship between the PK and the PD effect of BMS-354825, the PK was determined in TPTX rats in conjunction with evaluation of inhibition of serum calcium levels at 5, 15, and 30 mg/kg/dose. The rat blood was collected at 0, 2, 4, 7, and 24 hours on both day 1 and day 5 and the plasma levels of BMS-354825 were analyzed by HPLC/MS (FIG. 5). The PK parameters were derived and listed in Table 1. Following oral administration, BMS-354825 was rapidly absorbed with Tₘₐₓ of 2 hr for all three oral doses, which was consistent with previous PK studies in rodents conducted in house. The plasma level of BMS-354825 stayed higher at 24 hr than at 7 hr at 30 mg/kg which was also observed in previous PK studies in rats and was probably due to enterohepatic recycling. The exposure parameters, Cₘₐₓ and AUC_{0-24 hr}, appeared dose dependent on day 1 for 5, 15, and 30 mg/kg, respectively. On day 5, both Cₘₐₓ and AUC_{0-24 hr}, were less than those on day 1, which was also observed in previous toxicity study in rats. The decrease of systemic exposure was probably attributable to GI lesion caused by BMS-354825.

TABLE 1
Summary of pharmacokinetic parameters of BMS-354825

| | Treatment schedule | | | | | |
|---|---|---|---|---|---|---|
| | QD × 5; PO | | | | | |
| Dose (mg/kg/dose) | 5 | | 15 | | 30 | |
| Time (day) | Day 1 | Day 5 | Day 1 | Day 5 | Day 1 | Day 5 |
| Cmax (μM) | 0.65 | 0.23 | 0.54 | 0.08 | 0.16 | 0.06 |
| Tmax (hr) | 2.0 | 2.0 | 2.0 | 2.0 | 2.0 | 2.0 |
| AUC_{0-24 hr} (μM · hr) | 10.2 | 3.2 | 4.8 | 1.5 | 1.16 | 0.91 |

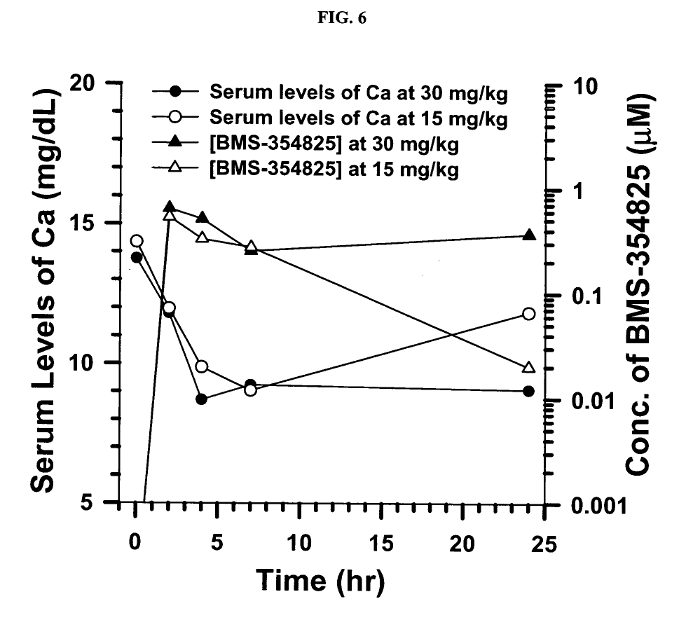

The plasma levels of BMS-354825 apparently correlated with the serum levels of calcium in TPTX rats at both 15 and 30 mg/kg/dose (FIG. 6). The maximum inhibition of serum calcium levels was achieved at 4 hr post drug administration, implying that the peak drug level in bone was probably several hours later than in plasma. Alternatively, the inhibition of serum calcium levels was a consequence of inhibition of bone resorption by BMS-354825, which could result in a delay between PK and PD effects. This kind of indirect PD response with several hour delay between Cₘₐₓ and Eₘₐₓ has been observed in previous studies in mice bearing tumor xenografts treated with BMS-354825 (Luo, F. R., Z. Yang, et al. (2005). Identification and Validation of a Pharmacodynamic Biomarker Assessing Target Exposure and Drug Efficacy for the Potent panSRC Kinase Inhibitor BMS-354825. AACR 96th Annual Meeting. Luo, F. R., Z. Yang, et al. (2004). Pharmacokinetics- and Pharmacodynamics-Guided Optimization of the Dose and Treatment Schedule for the Dual SRC/ABL Inhibitor BMS-354825t. ASH 46th Annual Meeting.).

In vitro Bone Resorption Assay

Fetal rat long bones were prepared and cultured as described previously. (Feyen J H M, Cardinaux F, Gamse R, Bruns C, Azria M, and Trechsel U. N-terminal truncation of salmon calcitonin leads to calcitonin antagonist. Structure activity relationship of N-terminally truncated salmon calcitonin fragments in vitro and in vivo. Biochemistry and Biophysiology Research Communication 1992; 187:8-13.) Briefly, pregnant Sprague Dawley rats were injected subcutaneously with 200 μCi ⁴⁵Ca on day 18 of gestation. The following day, radii and ulnae were dissected free from muscle and connective tissue. The cartilaginous ends of the bones were removed and the calcified diaphyses were cultured in 0.5 mL BGJ medium supplemented with 1 mg/mL bovine serum albumin (BSA, fraction V) in 24-well tissue culture plates in a CO₂ incubator at 37° C. overnight to reduce free exchangeable calcium. Next, medium was replaced and the bone explants were maintained in culture for 5 days in the presence or absence of the compounds to be tested. On day 2, a 100 μL aliquot was taken from the medium and the medium was replaced with fresh medium with or without treatments. On day 5 another 100 μL aliquot was taken from the medium. Residual ⁴⁵Ca was extracted from the bone explants by incubation in 200 μL 5% (w/v) trichloroacetic acid (TCA) for 24 hours and subsequently neutralized using 200 μL of 1N NaOH. The amount of radioactive calcium was determined using a Beckman liquid scintillation counter. Bone resorption was expressed as the percentage ⁴⁵Ca released by day 5 of total amount of ⁴⁵Ca originally incorporated in bone explants.

Serum Calcium in Thyro-Parathyroidectomized Rats

Thyro-parathyroidectomy (TPTX) surgery was performed on male Sprague-Dawley rats (200 gram body weight) as described previously.4 Thyro-parathyroidectomized (TPTX) rats with serum calcium values of 5-8 mg/dl were used for the study (normal reference range is 10-12 mg/dl). On the day of the study (48 hours post-surgery), blood was collected from the tail vein under isoflurane anesthesia, and Alzet mini-pumps (delivering 1 μl/hr) containing 0.3 μg/ml of parathyroid hormone (PTH) were implanted subcutaneously. Immediately following implantation of the pumps, the animals were dosed IP with vehicle, 5 IU salmon calcitonin or BMS-354825 at 3 & 10 mg/kg. Blood was collected at 3, 6 & 24 hours after dosing and analyzed for serum calcium levels.

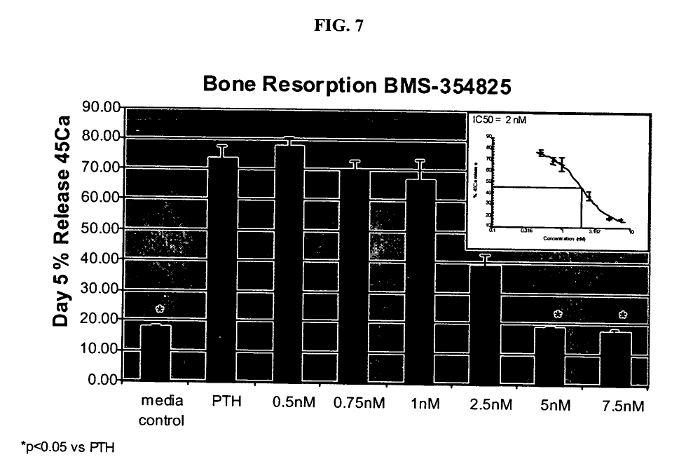

In vitro bone resorption assay: The results show that BMS-354825 is a potent inhibitor of bone resorption in vitro. PTH (10⁻⁸ M) stimulates bone resorption approximately 4-fold. BMS-354825 inhibits PTH-stimulated release of ⁴⁵Ca dose-dependently with an apparent IC₅₀ of 2 nM. At 5 nM BMS 354825 completely blocks PTH-stimulated bone resorption (see FIG. 7).

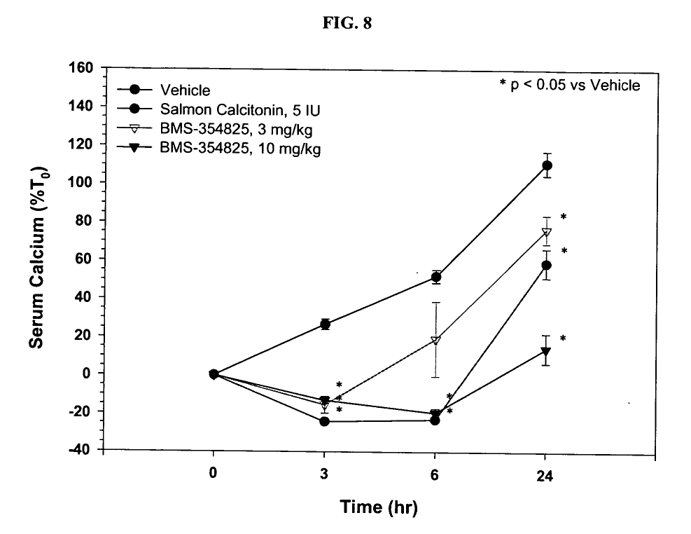

Serum calcium in thyro-parathyroidectomized rats: Results show that BMS-354825 is a potent inhibitor of bone resorption. Compared to vehicle treated animals, all treatments significantly prevented the PTH stimulated increase in serum calcium through the time course of this study. Due to high variability, the 3 hour time point in the 3 mg/kg BMS-354825 treated group was not significant. After 24 hours serum calcium levels of animals treated with BMS-354825 at 10 mg/kg are significantly lower than both vehicle and calcitonin (see FIG. 8 and Table 2).

TABLE 2

| | | Serum Calcium (mg/dl) | | | |
|---|---|---|---|---|---|
| Treatment | N | 0 Hour | 3 Hour | 6 Hour | 24 Hour |
| Vehicle, ip | 5 | 6.56 ± 0.18 | 8.32 ± 0.29 | 9.96 ± 0.22 | 13.84 ± 0.48 |
| Salmon | 5 | 6.68 ± 0.21 | 5.06 ± 0.13 * | 5.14 ± 0.15 * | 10.62 ± 0.64 * |
| Calcitonin, | | | | | |
| 5 IU ip | | | | | |
| BMS-354825, | 5 | 6.74 ± 0.29 | 5.68 ± 0.26 * | 7.94 ± 1.19 | 11.88 ± 0.61 * |
| 3 mg/kg ip | | | | | |
| BMS-354825, | 5 | 6.68 ± 0.25 | 5.80 ± 0.19 * | 5.36 ± 0.12 * | 7.70 ± 0.78 * |
| 10 mg/kg ip | | | | | |

p < 0.05 vs. Vehicle

What is claimed is:

1. A method of treating bone metastasis which comprises administering to a mammalian specie in need thereof a therapeutically effective amount of the compound of formula (I), 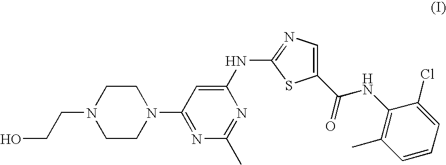
pharmaceutically acceptable salt, hydrate or solvate thereof.

2. A method of inhibiting hypercalcemia which comprises administering to a mammalian specie in need thereof a therapeutically effective amount of the compound of formula (I), 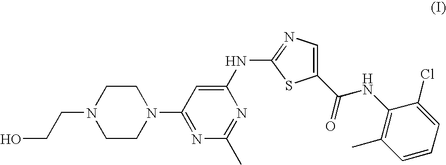
pharmaceutically acceptable salt, hydrate or solvate thereof.

3. A method of inhibiting bone resorption which comprises administering to a mammalian specie in need thereof a therapeutically effective amount of the compound of formula (I), 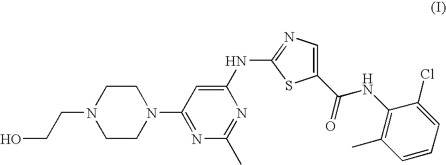
pharmaceutically acceptable salt, hydrate or solvate thereof.

4. A method of treating bone metastasis in breast cancer patients which comprises administering to a mammalian specie in need thereof a therapeutically effective amount of the compound of formula (I), 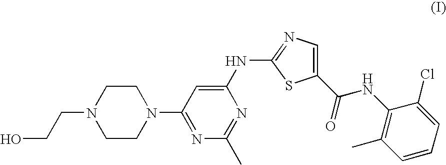
pharmaceutically acceptable salt, hydrate or solvate thereof.

5. A method of inhibiting hypercalcemia in breast cancer patients which comprises administering to a mammalian specie in need thereof a therapeutically effective amount of the compound of formula (I), 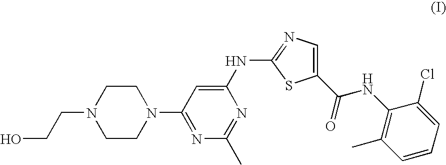
pharmaceutically acceptable salt, hydrate or solvate thereof.

6. A method of inhibiting bone resorption in breast cancer patients which comprises administering to a mammalian specie in need thereof a therapeutically effective amount of the compound of formula (I), 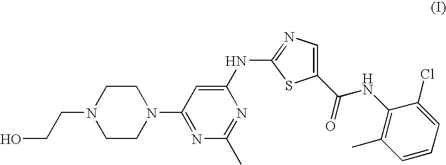
pharmaceutically acceptable salt, hydrate or solvate thereof.
